(19) United States
(12) Patent Application Publication
(10) Pub. No.: US 2018/0049831 A1
(43) Pub. Date: Feb. 22, 2018

(54) MEDICAL MANIPULATOR SYSTEM

(71) Applicant: OLYMPUS CORPORATION, Tokyo (JP)

(72) Inventors: Masatoshi IIDA, Tokyo (JP); Naoya HATAKEYAMA, Tokyo (JP)

(73) Assignee: OLYMPUS CORPORATION, Tokyo (JP)

(21) Appl. No.: 15/798,796

(22) Filed: Oct. 31, 2017

Related U.S. Application Data
(63) Continuation of application No. PCT/JP2015/063242, filed on May 8, 2015.

(51) Int. Cl. A61B 34/00; B25J 9/16; A61B 34/35; A61B 17/29

(52) U.S. Cl. CPC A61B 34/71; B25J 9/1689; A61B 34/25; A61B 34/35; A61B 34/74; A61B 2034/301; G05B 2219/35417; G05B 2219/39439; G05B 2219/45118; G05B 2219/49253; A61B 17/29

(57) ABSTRACT

A medical manipulator system including: a manipulator with a flexible insertion portion, a movable part at a distal end of the insertion portion, a detachable drive unit at a proximal end of the insertion portion, and a pair of tension transmission members transmitting a driving force generated in the drive unit to move the movable part; an input unit inputting an operation command for the manipulator; and a control unit controlling the drive unit based on the input operation command, the drive unit having a displacement sensor detecting displacement direction and amount of the tension transmission members and a tension-difference sensor detecting a tension difference between the tension transmission members, the control unit has a storage unit storing characteristics of the tension transmission members and a setting unit setting a control parameter for controlling the drive unit based on the characteristics and the detected tension difference, displacement amount and direction.

### CROSS-REFERENCE TO RELATED APPLICATIONS

This is a Continuation Application of International Application No. PCT/JP2015/063242 filed on May 8, 2015. The content of International Application No. PCT/JP2015/063242 is hereby incorporated herein by reference in its entirety.

### TECHNICAL FIELD

The present invention relates to a medical manipulator system.

### BACKGROUND ART

In flexible endoscopes in which a bendable portion is curved by means of wires, there is a known flexible endoscope that is provided with a rotary encoder for detecting the displacement amount and the displacement direction of wires and a tension sensor for detecting the tensions of the wires (for example, see PTL 1).

A drive unit that applies tensions to the wires is controlled in consideration of the displacement amount, the displacement direction, and the tensions of the wires, thereby avoiding a reduction in operability due to looseness of the wires.

### CITATION LIST

### Patent Literature

{PTL 1} Japanese Unexamined Patent Application, Publication No. 2000-300511

### SUMMARY OF INVENTION

According to an aspect of the present invention is a medical manipulator system including: a manipulator that is provided with a flexible insertion portion, a movable part that is provided at a distal end of the insertion portion, a drive unit that is detachably provided at a proximal end of the insertion portion and that generates a driving force, and a pair of tension transmission members that transmits the driving force generated in the drive unit by means of the tensions to move the movable part in forward and reverse directions; an operation-information input unit for inputting an operation command for the manipulator; and a control unit that controls the drive unit on the basis of the operation command input by the operation-information input unit, wherein the drive unit is provided with a displacement sensor that detects a displacement direction and a displacement amount of of the tension transmission members, and a tension-difference sensor that detects a tension difference between the pair of tension transmission members; and the control unit is provided with a storage unit that stores characteristics of the tension transmission members, and a parameter setting unit that sets a control parameter for controlling the drive unit on the basis of the characteristics stored in the storage unit, the tension difference detected by the tension-difference sensor, and the displacement amount and the displacement direction detected by the displacement sensor.

### BRIEF DESCRIPTION OF DRAWINGS

FIG. 1 is a view showing the overall configuration of a medical manipulator system according to an embodiment of the present invention.

FIG. 2 is a perspective view showing a manipulator provided in the medical manipulator system shown in FIG. 1.

FIG. 3 is a perspective view for explaining a drive unit provided in the manipulator shown in FIG. 2.

FIG. 4 is a graph showing the characteristics of a wire provided in the manipulator shown in FIG. 2.

FIG. 5 is a schematic view for explaining tensions applied to wires shown in FIG. 4.

FIG. 6 is a block diagram for explaining a control unit and the drive unit, which are provided in the medical manipulator system shown in FIG. 1, and the flow of a signal.

FIG. 7 is a block diagram showing a modification of the medical manipulator system shown in FIG. 6.

### DESCRIPTION OF EMBODIMENT

A medical manipulator system 1 according to an embodiment of the present invention will be described below with reference to the drawings.

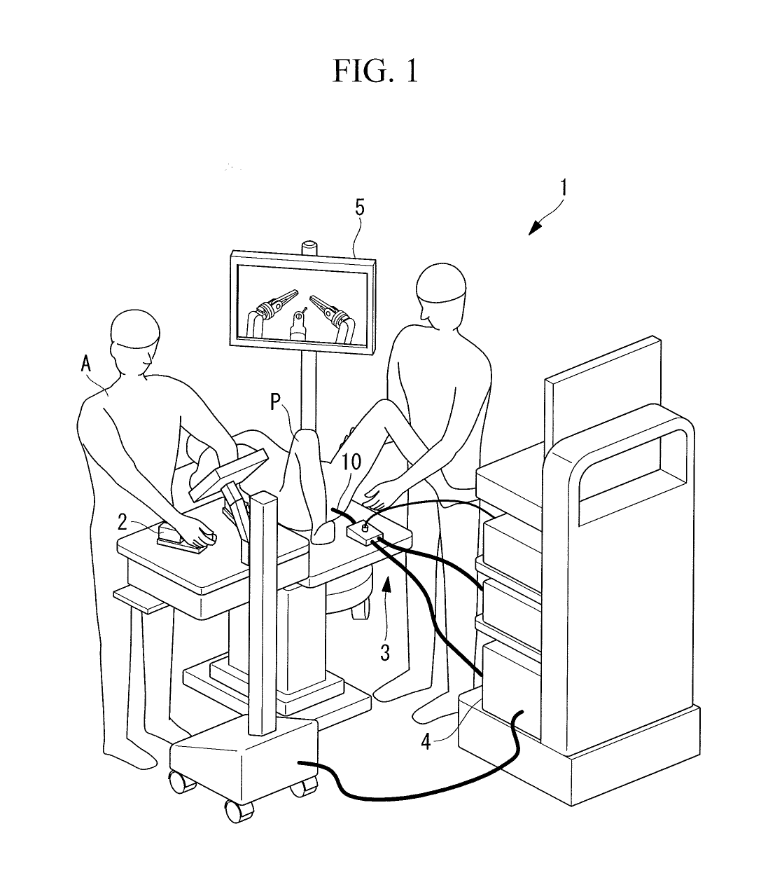

As shown in FIG. 1, for example, the medical manipulator system 1 of this embodiment is provided with: an operation input unit 2 that is operated by an operator A; a manipulator 3 that is inserted into a body cavity of a patient P; a control unit 4 that controls the manipulator 3; an operation-information input unit 24 for inputting an operation command for the manipulator 3 to the control unit 4 on the basis of an operation input that is input to the operation input unit 2; and a monitor 5.

The operation input unit 2 is an input device that has a lever (not shown) to be operated with the hand and fingers of the operator A and that has a substantially similar shape to and has the same axis structure as a movable part 6 disposed at a distal end of an insertion portion 10 of the manipulator 3, to be described later. Compared with the movable part 6, the operation input unit 2 has a scale ratio of about 10, for example. The operation input unit 2 need not have a similar shape to the movable part 6, and the scale ratio thereof is not limited to 10.

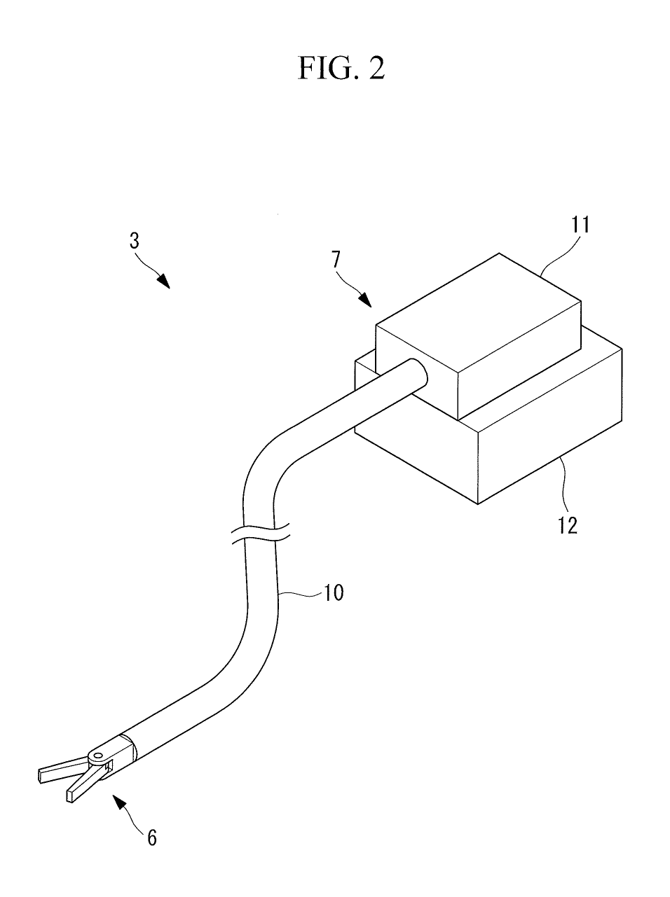

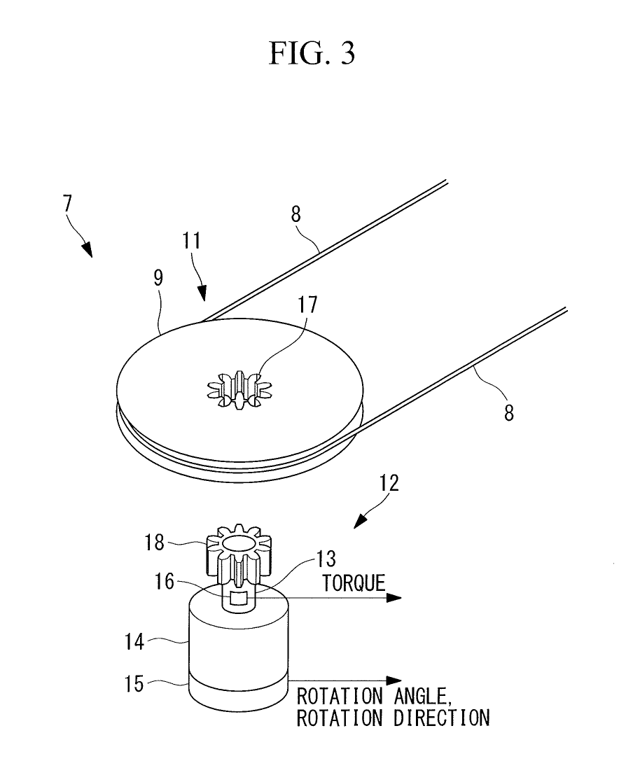

As shown in FIGS. 2 and 3, the manipulator 3 is provided with: the insertion portion 10, which is inserted into a body cavity of the patient P directly or via a channel for an endoscope inserted into the body cavity of the patient P or an overtube (not shown); the movable part 6, which is disposed at the distal end of the insertion portion 10; a drive device 7 that is disposed at a proximal end of the insertion portion 10; and wires (tension transmission members) 8 that connect the drive device 7 and the movable part 6 and that move the movable part 6 by means of the tensions thereof.

The movable part 6 is, for example, grasping forceps and is made to perform opening and closing movements by means of the tensions of the wires 8. In this embodiment, in order to simplify the explanation, a description will be given of a case in which the movable part 6 has a single axis. The movable part 6 may have a plurality of joints.

As shown in FIG. 3, the drive device 7 is provided with: a manipulator-side device 11 that includes a pulley 9 that is rotatably fixed to the proximal end of the insertion portion 10 and around which the wires 8 are looped; and a drive unit 12 that is provided so as to be capable of being attached to and detached from the manipulator-side device 11. Attachment/detachment of the drive unit 12 and the manipulator-side device 11 can be performed through spline coupling of the pulley 9 and a motor shaft 13, for example; however, the present invention is not limited thereto.

The drive unit 12 is provided with: a motor 14 that generates a rotational driving force; an encoder (displacement sensor) 15 that is fixed to the motor shaft 13 and that detects a rotation direction (displacement direction) and a rotation angle (the displacement amount) of the motor shaft 13; and a strain gauge (tension-difference sensor) 16 that is fixed to the motor shaft 13 and that detects, on the basis of the strain caused by twisting of the motor shaft 13, a torque (tension difference) that acts on the motor shaft 13.

Specifically, a spline shaft 18 that is provided on the motor shaft 13 is inserted into or removed from a spline hole 17 that is provided in the pulley 9, thereby making it possible to easily perform attachment or removal of the motor 14 and the pulley 9 through spline coupling.

Then, in a state in which the motor 14 is connected to the pulley 9, a torque having a magnitude that is calculated from the tension difference applied to the pair of wires 8 looped around the pulley 9 and the radius of the pulley 9 is applied to the motor shaft 13, thereby making it possible to detect the torque by using the strain gauge 16 and to easily calculate the tension difference from the torque.

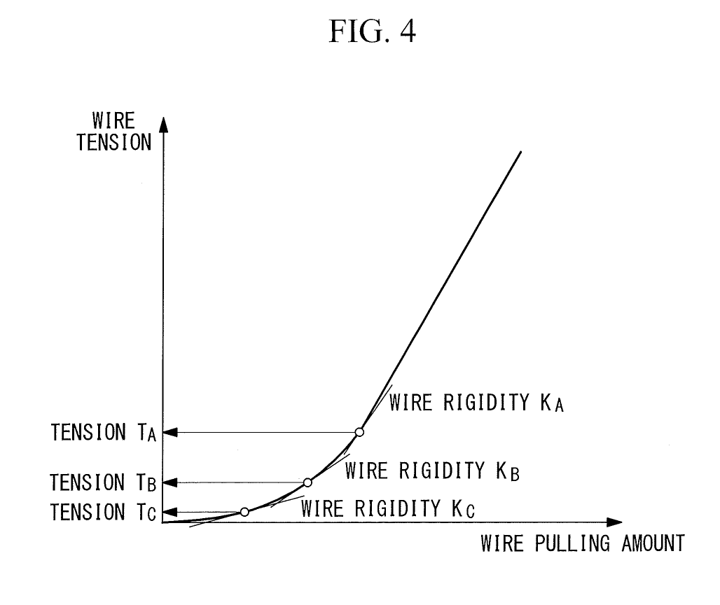

The wire 8 has the pulling-amount versus wire-tension characteristics shown in FIG. 4. Specifically, the rigidity of the wire 8 indicates the relationship between the pulling amount and the tension and has a non-linear characteristics, instead of an ideal linear characteristics. In a region where the tension is low, the tension is not large even when the pulling amount is increased, and, in a region where the tension is equal to or higher than a predetermined level, the magnitude of the pulling amount becomes proportional to the magnitude of the tension. The gradients of the characteristics shown in FIG. 4 at respective positions indicate the rigidities K_{A}, K_{B}, and K_{C} of the wire 8. In this case, the rigidities K_{A}, K_{B}, and K_{C} of the wire 8 are not at points but on gradients that are obtained by taking the difference between the values of tension differences at two points; thus, the gradients do not become zero even when the tension difference calculated from the torque becomes zero. Note that the pulling amount can be calculated from a parameter that is based on the rotation angle detected by the encoder 15, the pulley diameter, etc.

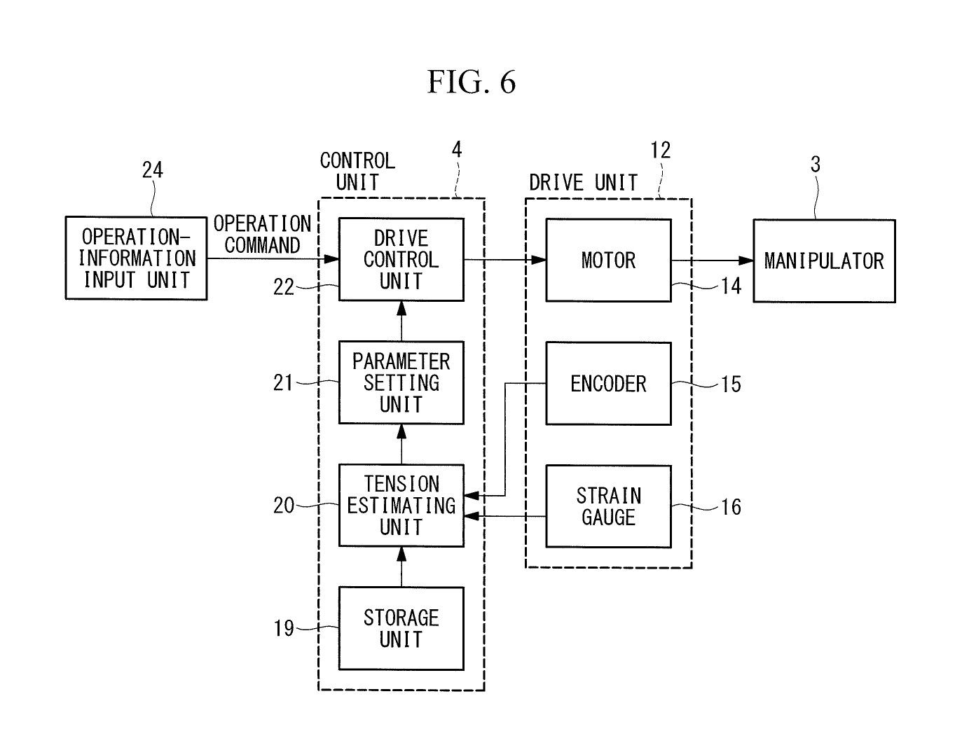

As shown in FIG. 6, the control unit 4 is provided with: a storage unit 19 that stores the above-described characteristics of the wire 8; a tension estimating unit 20 that estimates the tensions of the wires 8 on the basis of the torque detected by the strain gauge 16, the rotation angle and the rotation direction detected by the encoder 15, and the characteristics of the wire 8 stored in the storage unit 19; a parameter setting unit 21 that sets a control parameter on the basis of the tensions of the wires 8 estimated by the tension estimating unit 20; and a drive control unit 22 that controls the motor 14 by using the control parameter set by the parameter setting unit 21.

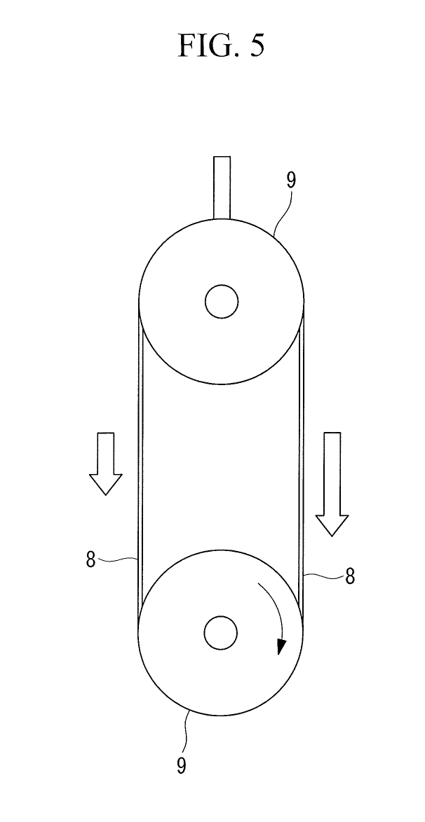

The tension estimating unit 20 calculates the rigidities of the wires 8 from the torque detected by the strain gauge 16 and the rotation angle detected by the encoder 15. Then, the tensions of the wires 8 are estimated by applying the calculated rigidities to the characteristics stored in the storage unit 19. The rotation direction detected by the encoder 15 is used to determine to which of the pair of wires 8 a large tension acts, as shown in FIG. 5. In the figure, a thin arrow indicates the rotation direction, thick arrows indicate directions in which the tensions of the wires 8 act, and the lengths of the thick arrows indicate the magnitudes of the tensions.

The parameter setting unit 21 sets a control parameter according to the tensions of the wires 8 estimated by the tension estimating unit 20.

As an example of the control parameter, a master-slave scale ratio can be used. The master-slave scale ratio is a parameter for determining the degree of the amount of movement of the movable part 6 with respect to the operation amount of the operation input unit 2.

In a case in which the insertion portion 10 of the manipulator 3 is extended straight, the master-slave scale ratio is set to 0.1, so that, when the lever of the operation input unit 2 is moved by 10 mm, the amount of movement of the movable part 6 can be 1 mm. Furthermore, for example, the master-slave scale ratio is set to 0.2, so that, when the lever of the operation input unit 2 is moved by 10 mm, the amount of movement of the movable part 6 becomes 2 mm. Thus, in order to achieve the same movement amount of 1 mm, the operator A just needs to move the lever of the operation input unit 2 by 5 mm.

Here, in a case in which the insertion portion 10 of the manipulator 3 is extended straight, the friction between a channel formed in the insertion portion 10 of the manipulator 3 and the wires 8 is the least, and a force exerted on the operation input unit 2 is directly transmitted to the movable part 6 as the tensions applied to the wires 8. Therefore, in this case, if the master-slave scale ratio is 0.1, this ratio coincides with the actual scale ratio between the operation input unit 2 and the movable part 6; thus, for example, an intuitive operation using the operation input unit 2 can be performed while confirming the movable part 6 on an endoscope image.

However, in a case in which the insertion portion 10 of the manipulator 3 is curved, friction between the channel formed in the insertion portion 10 and the wires 8 occurs at each bendable section, and the friction becomes larger as the degree of the curvature becomes larger. Then, as the friction becomes larger, the tensions applied to the wires 8 become larger.

Thus, a force exerted on the operation input unit 2 is unlikely to be transmitted to the movable part 6, and, if the master-slave scale ratio is still 0.1, even when the lever of the operation input unit 2 is moved by 10 mm, the amount of movement of the movable part 6 becomes less than 1 mm due to stretching of the wires 8.

Therefore, by storing the parameter setting unit 21, as the control parameter, a master-slave scale ratio that is increased as the tensions are increased, it possible to achieve the same movement amount of the movable part 6 by using the same operation command with respect to the operation input unit 2, regardless of the curved state of the insertion portion 10.

The operation-information input unit 24 inputs an operation command that is input when the operator A operates the operation input unit 2, to the drive control unit 22 of the control unit 4.

The operation of the thus-configured medical manipulator system 1 of this embodiment will be described below.

In order to treat an affected area located in a body cavity of the patient P by using the medical manipulator system 1 of this embodiment, the operator A inserts, into the body, the manipulator 3 from the movable part 6 disposed at the distal end thereof, while confirming an endoscope image or the like, and opposes the movable part 6 of the manipulator 3 to the affected area.

In this state, when the operator A operates the operation input unit 2 to input an operation command for the drive unit 12 to the operation-information input unit 24, the control unit 4 generates a control signal for controlling the drive unit 12 according to the operation command input from the operation-information input unit 24 and outputs the control signal to the drive unit 12.

When the motor 14 of the drive unit 12 is actuated to rotate the motor shaft 13, the rotation angle and the rotation direction of the motor shaft 13 are detected by the encoder 15, and a torque exerted on the motor shaft 13 is detected by the strain gauge 16.

Then, the tensions applied to the wires 8 are estimated by the tension estimating unit 20 on the basis of the detected torque and rotation angle. Furthermore, the parameter setting unit 21 sets a control parameter on the basis of the estimated tensions.

Specifically, through estimation of the tensions applied to the wires 8, the friction state of the wires 8, i.e., the curved state of the insertion portion 10, can be estimated. Therefore, there is an advantage that the control parameter, such as a master-slave scale ratio, is increased as the tensions become higher, thereby making it possible to precisely move the movable part 6 through an operation of the operation input unit 2 even when the degree of curvature of the insertion portion 10 is high, and thus, the tensions are difficult to be transmitted.

Then, according to the medical manipulator system 1 of this embodiment, because the tensions of the wires 8 are estimated without attaching a tension sensor to the wires 8, there is an advantage that, even if the insertion portion 10, which is inserted into the body, is formed to be disposable, an expensive sensor need not be disposed, thus making it possible to reduce the cost.

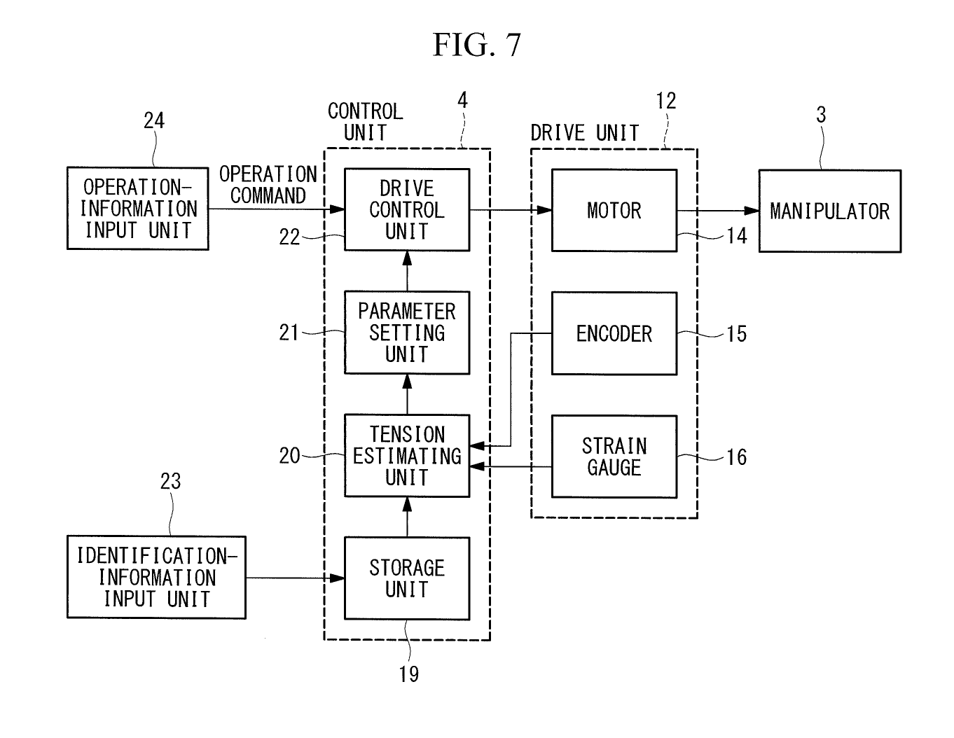

Note that, in the medical manipulator system 1 of this embodiment, although the characteristics of a single wire 8 are stored in the storage unit 19, instead of this, the characteristics of a plurality of wires 8 of a plurality of manipulators 3 may be stored in association with the identification information of the manipulators 3. In this case, as shown in FIG. 7, it is necessary to provide an identification-information input unit 23 for inputting, when the operator A operates the operation input unit 2, the identification information of the manipulator 3 on the basis of the operation input that is input to the operation input unit 2, and the characteristics of the wires 8 associated with the input identification information are read from the storage unit 19 and may be used for the estimation of the tensions.

The identification-information input unit 23 may input identification information on the basis of information input from the operation input unit 2, such as a keyboard, or may input identification information on the basis of information input from a barcode reader when a barcode is attached to the manipulator 3.

Furthermore, because the characteristics of the wire 8 stored in the storage unit 19 changes with use, it may also be possible to measure and update the characteristics of the wire 8 periodically or as needed.

Furthermore, the control parameter may be set during a calibration operation in response to an instruction from the operator A or may be set successively during the operation of the manipulator 3.

Furthermore, a calculated tension value may be compared with a predetermined threshold, and, when the tension value is lower than the threshold, or when the tension value is higher than the threshold, an alarm may be notified to the operator A. When the tension value is lower than the threshold, there is a possibility that the wire 8 is ruptured. Furthermore, when the tension value is higher than the threshold, there is a possibility that the movable part 6 is caught by external tissue etc.

From the above-described embodiments, the following aspects of the present invention are derived.

According to one aspect, the present invention provides a medical manipulator system including: a manipulator that is provided with a flexible insertion portion, a movable part that is provided at a distal end of the insertion portion, a drive unit that is detachably provided at a proximal end of the insertion portion and that generates a driving force, and a pair of tension transmission members that transmits the driving force generated in the drive unit by means of the tensions to move the movable part in forward and reverse directions; an operation-information input unit for inputting an operation command for the manipulator; and a control unit that controls the drive unit on the basis of the operation command input by the operation-information input unit, wherein the drive unit is provided with a displacement sensor that detects a displacement direction and a displacement amount of of the tension transmission members, and a tension-difference sensor that detects a tension difference between the pair of tension transmission members; and the control unit is provided with a storage unit that stores characteristics of the tension transmission members, and a parameter setting unit that sets a control parameter for controlling the drive unit on the basis of the characteristics stored in the storage unit, the tension difference detected by the tension-difference sensor, and the displacement amount and the displacement direction detected by the displacement sensor.

According to this aspect, the drive unit is attached to the proximal end of the insertion portion, the insertion portion is inserted into the body of a patient from the movable part side, the control unit controls, when an operation command is input by the operation-information input unit, the drive unit on the basis of the input operation command, a driving force generated in the drive unit is transmitted to the movable part by means of the tension transmission members in the form of tensions, and the movable part is actuated to perform treatment.

When the drive unit is actuated, the tension of one of the pair of tension transmission members is increased, and the movable part is moved so as to be pulled in a direction in which the tension is larger due to the tension difference.

When the insertion portion is curved, the tension transmission members passing therethrough are also curved, and the friction between the tension transmission members and the surrounding member is increased; therefore, a larger driving force is required to actuate the movable part.

According to this aspect, when the tension transmission members are displaced through the actuation of the drive unit, the displacement direction and the displacement amount are detected by the displacement sensor, and the tension difference acting on the pair of tension transmission members is detected by the tension-difference sensor; therefore, it is possible to estimate the state of the friction acting on the tension transmission members on the basis of the tension difference, the displacement amount, the displacement direction, and the characteristics of the tension transmission members.

Specifically, although the tension transmission members, such as wires, generally produce tensions due to elastic deformation, the rigidity, which indicates the relationship between the pulling amount and the tension, has a characteristics showing a non-linear relationship, instead of a characteristics showing an ideal linear relationship. Therefore, the rigidities of the tension transmission members are calculated from the tension difference, the displacement amount, and the displacement direction, and the state of the friction can be estimated by using this calculation results and the characteristics obtained in advance. Then, it is possible to cause the drive unit to generate a driving force for appropriately moving the movable part with respect to an operation input, on the basis of the estimated state of the friction.

In this case, because the displacement amount, the displacement direction, and the tension difference are detected by the displacement sensor and the tension-difference sensor, which are provided in the drive unit detachably attached to the proximal end of the insertion portion, a sensor for detecting tensions need not be provided in the insertion portion, which is provided with the tension transmission members, thus making it possible to reduce the cost even if the insertion portion is formed to be disposable.

In the above-described aspect, the parameter setting unit may calculate tensions acting on the tension transmission members on the basis of the tension difference detected by the tension-difference sensor and the displacement amount and the displacement direction detected by the displacement sensor, and may set the control parameter on the basis of the calculated tensions.

By doing so, it is possible to calculate the rigidities of the tension transmission members from the tension difference, the displacement amount, and the displacement direction and to easily calculate the tensions acting on the tension transmission members on the basis of the rigidities and the characteristics of the tension transmission members. Because the tensions, which cannot be directly detected in the drive unit side, are calculated from the tension difference and the displacement amount, which can be detected in the drive unit side, a sensor for detecting tensions need not be provided in the insertion portion, which is provided with the tension transmission members, thus making it possible to reduce the cost even if the insertion portion is formed to be disposable.

Furthermore, in the above-described aspect, the storage unit may store identification information of the manipulator and the characteristics of the tension transmission members in association with each other; the medical manipulator system may further comprise an identification-information input unit for inputting the identification information of the manipulator; and the control unit may read, from the storage unit, the characteristics of the tension transmission members associated with the identification information input by the identification-information input unit and may use the read characteristics of the tension transmission members for setting the control parameter.

By doing so, even when the characteristics of the installed tension transmission members are changed by exchanging the manipulator, because the characteristics stored in the storage unit in association with the identification information input by the identification-information input unit are read, it is possible to set an appropriate control parameter on the basis of the displacement amount, the displacement direction, and the tension difference of the tension transmission members, without depending on the individual difference of the manipulator.

According to the present invention, an advantageous effect is afforded in that a medical manipulator can be precisely controlled by setting an appropriate control parameter according to the tensions of wires, without attaching a tension sensor to the wires.

### REFERENCE SIGNS LIST

1 medical manipulator system
3 manipulator
4 control unit
6 movable part
8 wire (tension transmission member)
10 insertion portion
12 drive unit
15 encoder (displacement sensor)
16 strain gauge (tension-difference sensor)
19 storage unit
21 parameter setting unit
23 identification-information input unit
24 operation-information input unit
A operator

1. A medical manipulator system comprising:
a manipulator that is provided with a flexible insertion portion, a movable part that is provided at a distal end of the insertion portion, a drive unit that is detachably provided at a proximal end of the insertion portion and that generates a driving force, and a pair of tension transmission members that transmits the driving force generated in the drive unit by means of tensions to move the movable part in forward and reverse directions;
an operation-information input unit for inputting an operation command for the manipulator; and
a control unit that controls the drive unit on the basis of the operation command input by the operation-information input unit,
wherein the drive unit is provided with a displacement sensor that detects a displacement direction and a displacement amount of the tension transmission members, and a tension-difference sensor that detects a tension difference between the pair of tension transmission members, and
the control unit is provided with a storage unit that stores characteristics of the tension transmission members, and a parameter setting unit that sets a control parameter for controlling the drive unit on the basis of the characteristics stored in the storage unit, the tension difference detected by the tension-difference sensor, and the displacement amount and the displacement direction detected by the displacement sensor.

2. A medical manipulator system according to claim 1, wherein the parameter setting unit calculates tensions acting on the tension transmission members on the basis of the tension difference detected by the tension-difference sensor and the displacement amount and the displacement direction detected by the displacement sensor, and sets the control parameter on the basis of the calculated tensions.

3. A medical manipulator system according to claim 1,
wherein the storage unit stores identification information of the manipulator and the characteristics of the tension transmission members in association with each other,
the medical manipulator system further comprises an identification-information input unit for inputting the identification information of the manipulator, and
the control unit reads, from the storage unit, the characteristics of the tension transmission members associated with the identification information input by the identification-information input unit and uses the read characteristics of the tension transmission members for setting the control parameter.
